(19) United States
(12) Patent Application Publication
(10) Pub. No.: US 2008/0213334 A1
(43) Pub. Date: Sep. 4, 2008

(54) POLYELECTROLYTE MEDIA FOR BIOACTIVE AGENT DELIVERY

(76) Inventors: Nathan A. Lockwood, Minneapolis, MN (US); Joram Slager, St. Louis Park, MN (US); John V. Wall, Woodbury, MN (US); Peter H. Duquette, Edina, MN (US)

Correspondence Address: SURMODICS, INC., 9924 WEST 74TH STREET, EDEN PRAIRIE, MN, 55344, US

(21) Appl. No.: 11/863,545

(22) Filed: Sep. 28, 2007

Related U.S. Application Data
(60) Provisional application No. 60/848,422, filed on Sep. 29, 2006.

(51) Int. Cl. A61K 9/00; A61K 47/30; A61K 38/02; B05D 1/34; A61K 38/16; A61K 31/7052

(52) U.S. Cl. 424/423; 514/772.3; 514/2; 514/12; 514/44; 427/2.1; 427/426

(57) ABSTRACT

The invention provides polyelectrolyte hydrogels, blends, and multilayers for the controlled release of bioactive agents from implantable medical devices coated with or containing such media.

### CROSS-REFERENCE TO RELATED APPLICATION

The present non-provisional patent Application claims priority under 35 USC 119(e) from U.S. Provisional Patent Application having Ser. No. 60/848,422, filed on Sep. 29, 2006, and titled POLYELECTROLYTE MEDIA FOR BIOACTIVE AGENT DELIVERY; wherein the entirety of said provisional patent application is incorporated herein by reference.

### FIELD OF INVENTION

In one aspect, this invention relates to coating compositions for treating implantable devices with coatings for the controlled release of bioactive agents from the surface of the device. In another aspect, this invention relates to implantable gel matrices for the controlled release of bioactive agents from the matrix. In another aspect, this invention relates to methods for coating implantable devices with the coating compositions of the invention. In another aspect, this invention relates to methods for making bioactive agent delivery gel matrices.

### BACKGROUND OF THE INVENTION

Targeted drug delivery holds promise for many medical applications because it provides a mechanism by which a drug can be delivered directly to the site where it is needed, thus avoiding the toxic concentration of drugs necessary to achieve proper dosing when the drug is administered systematically.

Targeted delivery is particularly useful in surgical interventions where medical devices are implanted into the body of a patient or subject. However, placing a foreign object in the body can give rise to a number of deleterious side effects. These side effects not only compromise the patient's health; but can also compromise the function of the implanted device. Potential deleterious side effects include: infection at the implantation site, undesirable immunogenic responses, hyperplasia, and restenosis.

One approach to dealing with such undesirable side effects is to provide the surfaces of medical devices with coatings that render them more biocompatible. Consequently, significant effort is focused on the development of coatings for release of drugs from the surface of implanted articles. One method is to provide the device with an ability to deliver a bioactive agent at the implant site. For example, antibiotics can be released from the surface of the device to minimize infection or alternatively, antiproliferative drugs can be released to inhibit hyperplasia.

A number of drug delivery coatings have been described. See for example, U.S. Pat. No. 6,214,901; U.S. Pat. No. 6,344,035; U.S. Publication No. 2002-0032434; U.S. Publication No. 2002-0188037; U.S. Publication No. 2003-0031780; U.S. Publication No. 2003-0232087; U.S. Publication No. 2003-0232122; PCT Publication No. WO 99/55396; PCT Publication No. WO 03/105920; PCT Publication No. WO 03/105918; and PCT Publication No. WO 03/105919, which collectively disclose, inter alia, coating compositions having a bioactive agent in combination with a polymer component such as polyalkyl(meth)acrylate or aromatic poly(meth)acrylate polymer and another polymer component such as poly(ethylene-co-vinyl acetate) for use in coating device surfaces to control and/or improve their ability to release bioactive agents in aqueous systems. Other patents are directed to the formation of a drug containing hydrogel on the surface of an implantable medical device, these include Amiden et al, U.S. Pat. No. 5,221,698 and Sahatjian, U.S. Pat. No. 5,304,121. Still other patents describe methods for preparing coated intravascular stents via application of polymer solutions containing dispersed therapeutic material to the stent surface followed by evaporation of the solvent. This method is described in Berg et al., U.S. Pat. No. 5,464,650.

An emerging drug delivery coating utilizes polyelectrolyte multilayers (PEMs). Typically, PEMs are formed by layer by layer assembly (LBL), which allows for adsorption of layers of oppositely charged polyelectrolytes upon a surface. The technique is based upon electrostatic interactions between the oppositely charged polyelectrolytes.

Typically, in the LBL technique, PEMs are formed by the sequential adsorption of polyanionic and polycationic materials from dilute aqueous solutions onto a surface that has been pretreated to provide a charged surface onto which the first layer is absorbed. For example, if the surface is treated to render it positively charged, then the surface would first be dipped in a solution containing the polyanion. The surface is removed, dried and then dipped in a solution of the polycation and dried. The process is repeated until the desired number of layers is achieved.

Li et al. describe controlled delivery of therapeutic agents from medical devices coated with a PEM in U.S. Pat. No. 6,899,731 (the entire teaching of which is hereby incorporated by reference). The PEM of Li et al. is comprised of alternating layers of a negatively charged therapeutic agent and a cationic agent. Lynn et al. describe a PEM comprised of alternating layers of polyelectrolytes that carry an agent in U.S. patent application Ser. No. 10/280,268 (the entire teaching of which is hereby incorporated by reference). The agent is released by the sequential delamination of the alternating layers of polyelectrolytes.

The PEM drug delivery coatings described to date are non-ideal for a number of reasons. First, these PEM drug delivery coatings present hemocompatibility concerns. PEM coatings with a polycationic top layer will problematically present a positively charged surface at the implantation site. Positively charged surfaces are known to induce the formation of thrombi. Second, PEM coatings that are able to degrade may do so in an unpredictable manner (e.g., bulk degradation, delamination, etc.) making controlled drug release difficult if not impossible. Finally, the LBL assembly of PEMs is a time consuming and cost ineffective manufacturing process.

Despite the promise of the PEMs for drug delivery, there are problems that require resolution. There remains a need for biocompatible coatings that release a drug in a predictable manner and that can be manufactured in a cost effective and reproducible manner.

### SUMMARY OF THE INVENTION

Generally, the invention is directed to tunable or controllable release of bioactive agents from coatings provided on medical devices or from three dimensional matrices. The devices and matrices are implantable so that bioactive agents can be directed to specific sites within the body of a patient or subject.

According to some aspects, the invention is directed to polyelectrolyte compositions that can be used to form a number of different bioactive agent delivery media. The polyelectrolyte media comprise a first polyanion component and a second polycation component.

According to some embodiments, the polyanion and polycation components are selected so as to form as hydrogel. In some embodiments, the hydrogel forms a coating for a surface of a device. In other embodiments, the hydrogel forms a three dimensional matrix that can be implanted directly into a patient or subject or used to fill drug delivery devices.

According to other embodiments, the polyanion and polycation components are chosen to form an insoluble polyelectrolyte blend. This blend is distinguished from a hydrogel in that a blend does not absorb an appreciable amount of water. The blend can be used as a coating for a device.

Other embodiments provide methods for producing the polyelectrolyte bioactive agent delivery media. Some embodiments provide methods for spraying polyelectrolyte hydrogel and blend coatings. According to these methods, a spraying apparatus is provided that keeps the polyanion and polycation polymer component separate until the components are sprayed onto a surface.

Other aspects of the invention provide methods for treating patients or subjects with the polyelectrolyte bioactive agent delivery media.

### BRIEF DESCRIPTION OF THE DRAWINGS

FIG. 1 is a schematic side view of a coating apparatus according to an embodiment of the invention.

FIG. 2 is a schematic side view of a coating apparatus according to another embodiment of the invention.

FIG. 3 is a depiction of an elution profile of calcein from a medical device according to an embodiment of the invention.

FIG. 4 is a depiction of an elution profile of LHRH from a medical device according to an embodiment of the invention.

FIG. 5 is a depiction of an elution profile of BSA from a medical device according to an embodiment of the invention.

### DETAILED DESCRIPTION OF THE INVENTION

The invention will now be described with reference to the following embodiments. The embodiments described are exemplary only and are not intended to be exhaustive or to limit the invention to precise embodiments described. Rather, the embodiments are described and chosen only so that others skilled in the art can appreciate and understand the invention.

The invention is directed to polyelectrolyte media for delivery of a bioactive agent(s). The media of the invention can be used to coat the surfaces of devices. Other embodiments of the invention can be used to form three-dimensional matrices. Certain embodiments of the matrices are suitable for implantation at a treatment site. The terms “bioactive agent” and “drug” are used interchangeably. Also, the singular form of “agent” or “drug” is intended to encompass the plural forms as well.

The present invention is directed to methods and apparatuses for effectively treating a treatment site within a patient's body.

The invention is also directed to methods for applying the polyelectrolyte media to the surfaces of devices.

The inventive methods and apparatuses can be utilized to deliver bioactive agent to a treatment site in a controlled manner. The methods and apparatuses of the present invention can be used to provide flexibility in treatment duration as well as the type of bioactive agent delivered to the treatment site. In particular the present invention has been developed for controllably providing one or more bioactive agents to a treatment site within the body for a desired course of treatment.

The term “implantation site” refers to the site within a patient's body at which the implantable device is placed according to the invention. In turn a “treatment site” includes the implantation site as well as the area of the body that is to receive treatment directly or indirectly from a device component. For example, bioactive agent can migrate from the implantation site to areas surrounding the device itself, thereby treating a larger area than simply the implantation site.

Bioactive agent is released from the inventive media over time. The relationship between the amount of bioactive agent released from the inventive media and time can be plotted to establish a release or elution profile (cumulative mass of bioactive agent released versus time). Typically, the bioactive agent release profile can be considered to include an initial release of the bioactive agent and a release of the bioactive agent over time. The distinction between these two can often be simply the amount of time. The initial release is that amount of bioactive agent released shortly after the device is implanted. The release of bioactive agent over time includes the period of time commencing after the initial release.

The drug delivery media of the invention are formed in certain embodiments from polyelectrolyte first and second polymer components. In certain embodiments, the first and second polymers carry net charges that are opposite to each other. While in other embodiments, the media can be formed from one or more polymer components that carry both positive and negative charges along its length.

As used herein, the term “polyanion” refers to a polymer or substance that carries a net negative charge greater than one. Likewise, the term “polycation” refers to a polymer or substance that carries a net positive charge greater than one. The term polyampholyte refers to a polymer or other substance that carries both multiple positive and multiple negative charges.

The term “polyelectrolyte molecules” as used herein refers to polymers or other molecules that are polyanionic, polycationic, or polyampholytic.

As used herein the term “polyelectrolyte bioactive agent delivery media” refers to media that are formed from combinations of polyanion and polycations and/or polyampholytes.

The polyelectrolyte bioactive agent delivery media of the present invention can be formed from a diverse group of polyelectrolyte molecules, including, without limitation, synthetic polymers, including degradable and non-degradable; derivatized polymers, including the incorporation of photogroups (photoderivatization); natural polymers, both degradable and non-degradable, including polysaccharides (natural or modified), poly(amino acids), polynucleotides, proteins; linear polyelectrolytes; dendrimers; organic and inorganic nanoparticles; polyvalent low molecular weight organic compounds; and non-polymeric materials. A non-limiting list of polyelectrolyte materials is provided in Table I.

As will be appreciated, polyelectrolytes may be comprised of only positively charged or negatively charged groups or units. For example, a polycation may be comprised of only positively charged groups or units while a polyanion may be comprised of only negatively charged groups or units. Alternately, polyelectrolytes may be copolymers that have any combination of charged and/or neutral groups or units. For example, a polycation polyelectrolyte may be comprised of both neutral and positive groups or units. Likewise, a polyanion may be comprised of both neutral and negative groups. Alternately, a polycation or polyanion may be comprised of positive, negative, and neutral groups or units. The only requirement is that for a polycation, the net charge is positive and for a polyanion, the net charge is negative.

Polyelectrolyte materials can vary in molecular weight, charge density, hydrophobicity and hydrophilicity, flexibility, stereoregularity, and/or functional or charged group. In fact, varying these characteristic can advantageously modify properties of the polyelectrolyte drug delivery media of the present invention.

It will be appreciated by those skilled in the art, that polyanion and/or polycation polymers can be produced from any polymeric backbone by the addition of an appropriate number of charged groups to the backbone. Therefore, polymers carrying no net charge can be modified by chemical reaction so that they carry a charge. Additionally, weak polyelectrolytes can be strengthened, as desired, by the addition of appropriately charged groups. It will also be apparent that the polymers may initially carry no net charge, yet upon reaction to create polyelectrolyte drug delivery media of the invention, become charged through a variety of reaction mechanisms including, but not limited to, hydrolysis of ester groups to provide acid groups. Other modifications can be carried out by techniques known to those skilled in the art.

Polyelectrolytes can be modified to confer desired properties. For example, polyelectrolytes can be provided with photoreactive groups. Photoreactive groups have been described in detail in U.S. Pat. Nos. 4,973,493; 4,979,959; 5,002,582; 5,512,329; 5,414,075; and 5,714,360, the contents of which are hereby incorporated by reference.

Photoreactive species respond to specific applied external stimuli to undergo active specie generation with resultant covalent bonding to an adjacent chemical structure. The photoreactive species generate active species such as free radicals and particularly nitrenes, carbenes, and excited states of ketones upon absorption of electromagnetic energy. Exemplary photoreactive species include; aryl azides, acyl azides, azidoformates, sulfonyl azides, phosphoryl azides, diazoalkanes, diazoketones, diazoacetates, beta-keto-alpha-diazoacetates, aliphatic azo, ketenes, and photoactivated ketones and quinones.

As used herein, the term “photoderivatized polyelectrolyte” refers to polyelectrolytes that have been modified to carry such photogroups. A number of non-limiting examples of photoderivatized polymers are provided in Table I.

In some embodiments, photoreactive molecules provide the charge on the polymer. A number of charged photoreactive molecules have been described in detail in U.S. Pat. Nos. 5,714,360; 6,077,698; 6,278,018; 6,603,040; and 5,924,390, the contents of which are incorporated by reference.

TABLE I
Polyelectrolyte Materials

| | |
|---|---|
| CLASS | EXAMPLE |

NATURAL

| | |
|---|---|
| Viruses | |
| Lipids | |
| Liposomes | |
| Polyamino acids | Poly(lysine), poly(arginine), poly(glutamic |
| | acid), poly(aspartic acid) |
| Fibrous proteins | Collagen |
| Polysaccharides | Chitosan, xanathan, heparin, alginate, |
| | chondroitin sulfate, dextran sulfate, pectin |
| Modified polysaccharides | |
| Polynucleotides | DNA, RNA |

PHOTODERIVATIZED POLYMERS

Photo heparin
PA-AMPS-APMA
PA-AMPS-APMA-PEG
Photocollagen
PA-AEM-BBA
PVP-APMA
PA-AMPS-APMA-BBA
PA-BBA-APMA-BBA
Heparin-BBA-EAC-BBA
Poly-APMA
Poly-APMA-BBA-APMA
PA-APMA-BBA
PVP-AMPS-APMA-BBA
Polydimethylsiloxane-aminopropyl-BBA
PolydimethylAPMA-BBA-APMA-BBA
PolyMAPTAC-BBA-APMA-BBA
PVP-BBA-APMA-BBA-StearylDMAPMA
Quat
PolyHPMA-MAA-BBA-APMA-BBA
PA-AMPS-BBA-APMA-BBA
PA-Mal-EAC-lysine(alpha)-BBA-APMA-BBA
PA-Mal-EAC-lysine(epsilon)-BBA-APMA-BBA
PA-MAPTAC-BBA-APMA-BBA
PA-methacrylic acid-methoxyPEG1000MA-BBA-APMA-BBA
PA-Mal-EAC-NOS-APMA-BBA
DiBBE-DHBA-PA-APMA
DiBBE-DHBA-PA-AMPS

SYNTHETIC POLYELECTROLYTES

Poly(allyl amine hydrochloride)
Poly(ethyleneimine)
Poly(acrylamide)
Poly (diallyldimethyl-ammonium chloride)
Poly(vinylbenzyltrimethylamine)
Polyvinylpyridine
Poly(acrylic acid)
Poly(vinylsulfate)
Poly(methacrylic acid)
Poly(styrene sulfonate)
Poly(maleic acid)
Poly(fumaric acid)

In certain embodiments, controllable drug delivery is accomplished with the use of polyelectrolyte hydrogels or gels. The terms “hydrogels” and “gel” are used interchangeably. In these embodiments, the polyelectrolytes are selected so that when combined in the appropriate ratios, a hydrogel forms. The polyelectrolytes can be polymeric in nature or can be selected from non-polymeric materials, examples of which are provided in Table 1. Thus in some embodiments, the hydrogels are formed from a first polyelectrolyte polymer component and a second polyelectrolyte polymer component.

Typically, polyelectrolyte hydrogels form a matrix that is crosslinked by electrostatic interactions between the opposite charges present on the polyelectrolytes. Hydrogels are characterized by insolubility in water, their ability to absorb a significant amount of water to confer a jelly-like consistency to the hydrogel, and are often mechanically deformable. Thus in some embodiments, the hydrogels are crosslinked via the electrostatic interactions between the charged groups. In other embodiments, additional crosslinking may be provided e.g., by covalent or additional ionic crosslinking.

The characteristics of the hydrogel can be manipulated in a number of manners. First, the polyelectrolyte components can be varied with respect to functional group, charge density, molecular weight, flexibility, hydrophobicity and hydrophilicity, and stereoregularity. Characteristics can also be manipulated by regulating the conditions under which the hydrogel is formed. For example, pH, ionic strength of solvent, concentration, temperature, and mixing. (See Dumitriu et al. 1998, the entire content of which is incorporated by reference).

Polyelectrolyte materials can be selected or matched for use for delivery of specific bioactive agents. For example, alginate and polyethyleneimine polymers are known protein stabilizers. Protein stabilization is particularly important since the function of proteins or peptides is often dependent on quaternary structure. Protein stabilizing polyelectrolyte material can thus be selected in situations where protein-based bioactive agents are to be delivered. Other factors can be considered to specifically tailor a bioactive agent delivery gel or any other polyelectrolyte medium described herein, to the particular bioactive agent. For example, in some embodiments, polyelectrolytes are selected so as to form degradable hydrogels that will dissolve and be removed when implanted in vivo. For example, in some embodiments collagen and alginic acid form a degradable hydrogel. In other embodiments, polymers are selected so as to form non-degradable hydrogels.

In some embodiments, polyelectrolyte hydrogels are formed by mixing polyanionic and polycationic materials together in one solution. In other embodiments, separate solutions of the polyanion and polycation are mixed together. In these embodiments, better control of the gel set up time is achieved. Controlled gel set-up time is particularly useful in applications where the hydrogel will be used to fill three dimensional spaces in devices.

Advantageously, the gel set up time can be manipulated by the selection of specific polyelectrolyte materials and the ratio of polyanion to polycation. For example, in some embodiments, the level of substitution of the polyanion is used to control gel times. For faster gel set up times, a polymer with a higher degree of ethylene substitution is used. In other embodiments, the relative ratio of polyanion is manipulated. For example, to reduce the gel set up time, the polyanion concentration is increased. In circumstances where extended gel set up times are desirable, the polyanion concentration is decreased. In other embodiments, gel time is manipulated by controlling the specific polymer used, i.e., the level of substitution and the polyanion to polycation ratio. In these embodiments, gel times are reduced by increasing both the level of substitution of the polycation and the concentration of the polyanion. Likewise, gel time can be increased by decreasing substitution and the polyanion concentration.

Properties of the hydrogels can be advantageously controlled by selection of polyanion and polycations and/or their relative ratios. For example, biodegradable polymers are selected in embodiments where a biodegradable hydrogel is produced. In other embodiments, polyanions and/or polycations with photogroups are selected to form hydrogels capable of coupling bioactive agents or other substances. Such bioactive agent may improve the biocompatibility of the hydrogel and/or may elicit a desired physiological response. The use of such photogroups is described in U.S. Pat. Nos. 4,973,493; 4,979,959; 5,002,582; 5,512,329; and 4,973,493.

The polyelectrolyte hydrogels can be further stabilized by enhancing the ionic interactions between the opposite charges on the polyelectrolyte materials. In these embodiments, a protein or peptide with an accessible charged species is incorporated into the hydrogel. The protein or peptide may be the bioactive agent intended for release from the hydrogel. This embodiment exemplifies the synergistic relationship between the protein or peptide and the hydrogel, with the ionic interactions stabilizing the protein while simultaneously stabilizing the hydrogel/protein or peptide complex.

In some embodiments, polyampholytes are selected to form the hydrogels of the invention. Polyampholytes are polyelectrolytes that contain both positive and negative charges along their length. In these embodiments, the polyampholyte serves as both the polyanion and polycation. Polyampholytes provide ionic interactions between the negative and positive charge groups along their length similar to those between charged groups provided on separate polyelectrolyte molecules.

In some embodiments the hydrogels are used to coat at least a portion of a surface a medical device. In these embodiments, the bioactive agent delivery medium is referred to as a “hydrogel coating”.

In other embodiments, the polyelectrolyte hydrogel is used to produce a three dimensional bioactive agent delivery matrix. These matrices may be implanted into subjects or patients for delivery of a bioactive agent(s). In some embodiments, the hydrogel matrix is implanted directly into the subject or patient. In other embodiments, the hydrogel may be formed in situ. In yet other embodiments, the hydrogels may be used to fill hollow interiors of drug delivery devices that are implanted in a subject or patient.

In embodiments where the hydrogel is used to fill hollow interiors, control over the rate of gel set up is particularly useful. Thus, in some embodiments, the rate of gel set up is controlled so that the polyanion and polycation can be mixed together outside of the device with the hollow interior. In this case, the gel set up time is extended so that the mix of the polyanion and polycation remains fluid for sufficient amount of time so that it can be easily delivered to the hollow interior.

As will be appreciated a number of other properties of the hydrogels described can be modified. For example, a number of properties can be manipulated by techniques described with respect to any embodiment described herein or by other techniques within the knowledge of those skilled in the art.

In some embodiments, controllable drug delivery is accomplished by providing polyelectrolyte bioactive agent delivery media that comprise polyanionic and polycationic polymers that form insoluble precipitates when mixed. As used herein, such mixtures are referred to as “polyelectrolyte blends” or “blends”. Such blends can be distinguished from polyelectrolyte hydrogels and polyelectrolyte multilayers. Polyelectrolyte multilayers are composed of alternating and discrete layers of polyanion and polycation. Polyelectrolyte hydrogels are mixtures of polyanions and polycations that are capable of absorbing significant amounts of water. In the blends of the present invention, the polyanion and polycation are not provided in separate layers but rather are intermingled and associated through electrostatic interactions between the opposite charges on the polyanion, polycation, or polyampholyte and do not absorb an appreciable amount of water.

The blends of the present invention can be applied to surfaces as coatings. For example, the polyelectrolyte blend can be applied to surfaces of medical devices that will be implanted into the body of a patient or subject. In these embodiments, the polyelectrolyte material is referred to as a blend coating.

As is evident, certain advantages are achieved through the use of polyelectrolyte blends or hydrogels. For example, the net charge of a blend or hydrogel can be controlled as compared to polyelectrolyte multilayers. Due to layered nature of PEMs, any surface coated with a PEM will present a net charge to the environment in which it implanted. Undesirable hematological responses can occur in circumstances where a coating with a net positive charge is in contact with blood. The polyelectrolyte blends and hydrogels of the present invention avoid this potential problem since the net charge of the blend or hydrogel is controllable.

As with the hydrogels described above certain characteristics can be achieved by selecting appropriate polyelectrolyte materials to form the blend. Such characteristics include biodegradability. For example, in some embodiments, biodegradable polyelectrolyte materials are selected to produce degradable blends. For example, in some embodiments, a degradable blend is formed from poly(lysine) and poly(aspartic acid). In other embodiments, non-degradable materials are selected to produce non-degradable blends. For example, in some embodiments, non-degradable blends are formed from synthetic poly(styrene sulfonate) and poly(allyl amine hydrochloride).

As with all of the media described, blends can be produced from natural polyelectrolyte polymers. For example, in some embodiments, blends are formed from polylysine and DNA. In yet other embodiments, the blend is formed from chitosan and heparin.

As described with respect to the hydrogel embodiments, polyelectrolyte materials can be modified. Therefore, in some embodiments, the polyelectrolyte materials are modified to confer specific properties. For example, the materials can be photoderivatized so that the blends contain photoreactive species.

As will be appreciated, a number of other properties of the blend can be modified. For example, a number of properties can be manipulated by techniques described with respect to any embodiment described herein or by other techniques within the knowledge of those skilled in the art.

The ratio of polyanion to polycation determines the net charge within the microenvironment of any particular polyelectrolyte blend or gel. As used herein, the term “microenvironment” refers to the environment, formed by the polyelectrolyte media, to which the bioactive agent is exposed. According to some aspects, the net charge of the microenvironment is controllable so that the pH of the microenvironment can be regulated.

As already described, polyelectrolytes include a number of charged residues or groups. As a practical matter, not all of the charged groups become involved in the electrostatic interactions that occur between oppositely charged groups on the polyelectrolytes of the media of the invention. The groups that are not involved in the electrostatic interactions are referred to as non-participating groups.

Non-participating charged groups contribute to the overall charge of the microenvironment of the blend or hydrogel. In some cases, particularly when one polyelectrolyte is provided in excess, entire polyelectrolyte molecules will not participate in electrostatic interactions. In these cases, it is theorized that at least some of the non-participating molecules will become entrapped in the blend or hydrogel media and contribute to the overall charge of the microenvironment.

Thus, according to some aspects, the pH of the microenvironment is controlled by stoichiometric considerations regarding the charged residues themselves and/or the relative ratio of polyanion to polycation. For example, an excess of negatively charged groups can be provided by selecting or engineering a polyanion that when combined with a polycation to form a medium, supplies non-participating negatively charged groups. These excess, non-participating negatively charged groups will impart a residual negative charge to blend or hydrogel microenvironment. It is understood that excess positively charged groups can be provided to impart a residual positive charge to the microenvironment.

In other embodiments, excess charged groups are provided by supplying either the polyanion or polycation in sufficient excess (dependent upon the desired residual charge) so that the net number of charged groups outnumbers the net number of oppositely charged groups. In this alternative, depending upon the chemical characteristics of the particular polyelectrolytes selected, the residual charge is imparted by non-participating charged groups or from charged groups on nonparticipating molecules that are entrapped in the gel or blend.

In certain embodiments, blends or gels with no net charge are provided. In these embodiments, polyelectrolytes are selected or engineered so that the ratio of positively charged groups to negatively charged groups is substantially 1:1. While in other embodiments, blends with a net positive or net negative charge are provided in order to regulate the pH of the blend or hydrogel microenvironment.

The net charge of the microenvironment of the blend or gel can be manipulated so that the pH is suitable for the specific application. For example, as is well known, protein stability is highly pH-dependent. Thus, in embodiments where a protein-based or other pH-susceptible bioactive agent is employed, the microenvironment of the blend or hydrogel in which the agent is incorporated can be specifically tuned to stabilize the bioactive agent.

It will be appreciated by those skilled in the art, that other techniques to control pH must be employed when non-acidic and/or non-basic polyelectrolyte materials are employed. Thus, in some embodiments the pH of the microenvironment is controlled by protocols well known to those skilled in the art.

The pH-dependence of a number of proteins is well known. For example, certain proteins, such as BSA, are acid-labile. When acid-labile bioactive agents are implemented, the hydrogel or blend can be engineered to minimize the acidity of the microenvironment by decreasing the net negative charge by the methods described above. Microenvironment conditions suitable for any particular bioactive can be determined by those with skill in that art, without undue experimentation.

Regulation of the pH of the microenvironment can also be used to control the elution profile of a bioactive agent. Certain microenvironment conditions can retard release of the bioactive agent. For example, pH-dependent protein denaturation can expose hydrophobic regions and resultant aggregation. Aggregation can obstruct release of the protein from the media. Likewise, in embodiments in which the bioactive agent carries a net charge, a microenvironment with a like net charge will impede the diffusion rate and thus impact the elution profile. Thus, according to some aspects, bioactive agent release is manipulated by controlling the net charge or pH of the microenvironment of the hydrogel or blend in which the bioactive agent is incorporated. As will be appreciated, in addition to ensuring release of bioactive agent, pH considerations can be used to fine tune the elution profile of any particular bioactive agent.

In some embodiments, controllable drug delivery is provided by incorporating bioactive agents into polyelectrolyte multilayer (PEM) coatings. The term “PEMs”, as used herein, refers to at least one layer of polyanion and at least one layer of polycation immediately adjacent to the polyanion layer. PEMs are comprised of alternating layers of polyanion and polycation. In these embodiments, polyelectrolyte materials are selected to confer the desired properties to the PEM. For example, in some embodiments, biodegradable PEMs are provided, while in others, non-degradable PELs are provided. The PEM coating can be tailored for specific applications by selection of appropriate polyelectrolyte materials.

In some embodiments, the polyelectrolytes are selected from natural polymers while in others one or both of the polyanion and/or polycation are selected from synthetic polymers. For example, in one embodiment, the polycation is polyethyleneimine (PEI) and the polyanion is polyacrylic acid (PAA). In this embodiment, the PEM is comprised of the alternating and discrete layers of PEI and PAA.

In yet other embodiments, photoderivatized polycation and polyanion polymers are chosen. In these embodiments, one of the polycation or polyanion is provided with photogroups. In other embodiments, both polycation and polyanion are photoderivatized.

The inclusion of photo-polyelectrolytes advantageously can be used to provide additional crosslinking between the polycation and polyanion to further stabilize the polyelectrolyte drug delivery medium. Additionally, photogroups can be used to facilitate attachment of the polyanion and\or polycation to a surface.

In other embodiments, the bioactive agent itself forms either the polyanionic or polycationic layer of the PEM. For example, in some embodiments, the PEM is formed of a polyanionic polymer and a negatively charged bioactive agent. Conversely, in other embodiments, the PEM is formed of a polycationic polymer and a positively charged bioactive agent. In these embodiments, the polymeric component can be specifically selected to produce a coating with desired characteristics. For example, a photoderivatized, degradable, or non-degradable polyelectrolyte can be selected.

As will be appreciated, a number of other properties of the PEMs can be modified. For example, a number of properties can be manipulated by techniques described with respect to any embodiment illustrated herein or by other techniques within the knowledge of those skilled in the art.

To form a PEM, at least one layer of each of the polyanion and polycation is required. In some embodiments, the PEM is comprised of one layer each of polyanion and polycation. In other embodiments, multiple layers of polyanion and polycation are provided. As will be appreciated, any number of layers is possible. The number of layers is selected dependent on a number of factors, including, but not limited to, desired thickness of the coating.

In some embodiments, additives are provided to the polyelectrolyte media. Such additives can used in conjunction with the hydrogel coatings and three dimensional matrices, blend coatings and multilayers described herein.

Additives can be classified into two groups; those that affect release rate of a bioactive agent and those that affect properties of the polyelectrolyte medium itself. Both types are encompassed within the scope of the present invention.

In some embodiments, properties of the polyelectrolyte media are modified by the addition of crosslinking agents. A non-limiting example includes photoreactive crosslinking agents. Such crosslinking agents have been described in detail in U.S. Pat. Nos. 5,414,075; 5,637,460; 5,714,360; 6,077,698; 6,278,018; 6,603,040; and 6,924,390, the entire contents of which are incorporated by reference. Such crosslinking agents can be used, for example, to modify the strength of the polyelectrolyte medium. The crosslinking agents provide additional crosslinking between the polyanion and the polycation. Such crosslinking may be stronger than the electrostatic crosslinking typically found in polyelectrolytes. Thus, chemical crosslinking agents are used to manipulate the strength of the bioactive agent delivery media.

In other embodiments, divalent cations are added to provide additional electrostatic crosslinking. Divalent cations can impact both the characteristics of the hydrogel itself as well as the elution profile of the bioactive agent from the hydrogel. For example, divalent cations, such as, for example, Ca²⁺ can be added to any hydrogel embodiment. Without intending to be bound by theory, it is believed that the divalent cation provides additional electrostatic crosslinking between the polyelectrolytes. Such crosslinking not only strengthens the hydrogel, but also impacts the elution profile of a bioactive agent therefrom. Thus, in some embodiments, the elution profile of the bioactive agent is modulated by the use of divalent cations.

In other embodiments, crosslinking agents are used to modify properties specific to the biocompatibility of the polyelectrolyte media. Typically, the media of the present invention will be implanted into the bodies of patients and subjects. In general, it is desirable that the media not induce reactions that are undesirable for the particular application in the body such as blood clotting, tissue death, tumor formation, allergic reaction, foreign body reaction (rejection) or inflammatory reaction. Generally, adverse reactions are avoided by specifically selecting biocompatible polyelectrolytes. However, crosslinking agents can be used to further enhance or modify the biocompatibility of any particular polyelectrolyte bioactive agent delivery medium. For example, heparin can be crosslinked to the medium to prevent the formation of blood clots in circumstances where the medium will contact blood and the formation of blood clots is not desirable. Those skilled in the art will recognize that any number of molecules can be crosslinked to confer any number of specific properties.

In other aspects, additives can be included to impact the release of the bioactive agent from the media. Suitable additives include, but are not limited to, hydrophobic molecules, hydrophilic antioxidants, and excipients. Illustrative excipients include salts, polyethylene glycol (PEG) or hydrophilic polymers, and acidic compounds. Alternatively, additives can be included to impact imaging of the media once it is implanted.

Buffers, acids, and bases can be incorporated in the polyanion and/or the polycation to adjust their pH. Such additives can be used to increase the strength of the charge on the polyelectrolyte. Regulation of pH not only can be used to modify the release rate of the bioactive agent, but also to stabilize the bioactive agent.

The term “bioactive agent”, as used herein, will refer to a wide range of biologically active materials or drugs that can be incorporated into a drug delivery medium of the present invention. Bioactive agents useful according to the invention include virtually any substance that possesses desirable therapeutic characteristics.

It will be understood that the invention can provide any number of bioactive agents. Thus, reference to the singular form of “bioactive agent” is intended to encompass the plural form as well.

Exemplary bioactive agents include, but are not limited to, peptide, protein, carbohydrate, nucleic acid, lipid, polysaccharide or combinations thereof or synthetic or natural inorganic or organic molecule, that causes a biological effect when administered in vivo to an animal, including but not limited to birds and mammals, including humans. Nonlimiting examples are antigens, enzymes, hormones, receptors, peptides, and gene therapy agents. Examples of suitable gene therapy agents include a) therapeutic nucleic acids, including antisense DNA and antisense RNA, and b) nucleic acids encoding therapeutic gene products, including plasmid DNA and viral fragments, along with associated promoters and excipients. Examples of other molecules that can be incorporated include nucleosides, nucleotides, vitamins, minerals, and steroids.

Drug delivery media prepared according to this invention can be used to deliver drugs such as nonsteroidal anti-inflammatory compounds, anesthetics, chemotherapeutic agents, immunotoxins, immunosuppressive agents, steroids, antibiotics, antivirals, antifungals, steroidal antiinflammatories, anticoagulants, antiproliferative agents, angiogenic agents, and anti-angiogenic agents. In some embodiments, the bioactive agent to be delivered is a hydrophobic drug having a relatively low molecular weight (i.e., a molecular weight no greater than about two kilodaltons, and optionally no greater than about 1.5 kilodaltons). For example, hydrophobic drugs such as rapamycin, paclitaxel, dexamethasone, lidocaine, triamcinolone acetonide, retinoic acid, estradiol, pimecrolimus, tacrolimus or tetracaine can be included in the media and are released over several hours or longer.

Classes of medicaments which can be incorporated into the media of this invention include, but are not limited to, anti-AIDS substances, antineoplastic substances, antibacterials, antifungals and antiviral agents, enzyme inhibitors, neurotoxins, opioids, hypnotics, antihistamines, anti-diabetics (e.g., rosiglitazone), immunomodulators (e.g., cyclosporine), tranquilizers, anticonvulsants, muscle relaxants and anti-Parkinsonism substances, antispasmodics and muscle contractants, miotics and anticholinergics, immunosuppressants (e.g. cyclosporine), anti-glaucoma solutes, anti-parasite and/or anti-protozoal solutes, antihypertensives, analgesics, antipyretics and anti-inflammatory agents (such as NSAIDs), local anesthetics, ophthalmics, prostaglandins, anti-depressants, antipsychotic substances, antiemetics, imaging agents, specific targeting agents, neurotransmitters, proteins, and cell response modifiers. A more complete listing of classes of medicaments may be found in the Pharmazeutische Wirkstoffe, ed. A. Von Kleemann and J. Engel, Georg Thieme Verlag, Stuttgart/New York, 1987, incorporated herein by reference.

Antibiotics are recognized as substances which inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, tobramycin, erythromycin, quinolones (including but not limited to ciprofloxacin), cephalosporins, geldanamycin and analogs thereof. Examples of cephalosporins include cephalothin, cephapirin, cefazolin, cephalexin, cephliadine, cefadroxil, cefamandole, cefoxitin, cefaclor, cefuroxime, cefonicid, ceforanide, cefotaxime, moxalactam, ceflizoxime, ceftriaxone, and cefoperazone.

Antiseptics are recognized as substances that prevent or arrest the growth or action of microorganisms. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Antiviral agents are substances capable of destroying or suppressing the replication of viruses. Examples of antiviral agents include methyl-p-adamantane methylamine, hydroxyethoxymethylguanine, adamantanamine, 5-iodo-2′-deoxyuridine, trifluorothymidine, interferon, and adenine arabinoside.

Enzyme inhibitors are substances which inhibit an enzymatic reaction. Examples of enzyme inhibitors include edrophonium chloride, N-methylphysostigmine, neostigmine bromide, physostigmine sulfate, tacrine HCl, tacrine, 1-hydroxymaleate, iodotubercidin, p-bromotetramisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−), deprenyl HCl, D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylyxanthne, papaverine HCl, indomethacin, 2-cyclooctyl-2-hydroxyethylamine hydrochloride, 2,3-dichloro-alpha-methylbenzylamine, 8,9-dichloro-2,3,4,5-tetrahydro-1H-2-benzazepine hydrochloride, p-aminoglutethimide, p-aminoglutethimide tartrate, R(+), p-aminoglutethimide tartrate, S(−), 3-iodotyrosine, alpha-methyltyrosine, L(−), alpha-methyltyrosine, DL(−), cetazolamide, dichlorphenamide, 6-hydroxy-2-benzothiazolesulfonamide, and allopurinol.

Antipyretics are substances capable of relieving or reducing fever. Anti-inflammatory agents are substances capable of counteracting or suppressing inflammation. Examples of such agents include aspirin (acetylsalicylic acid), indomethacin, sodium indomethacin trihydrate, salicylamide, naproxen, colchicine, fenoprofen, sulindac, diflunisal, diclofenac, indoprofen and sodium salicylamnide.

Local anesthetics are substances which inhibit pain signals in a localized region. Examples of such anesthetics include procaine, lidocaine, tetracaine and dibucaine.

Imaging agents are agents capable of imaging a desired site in vivo. Examples of imaging agents include substances that have a detectable label e.g., antibodies attached to fluorescent labels. The term antibody includes whole antibodies or fragments thereof.

Cell response modifiers are chemotactic factors such as platelet-derived growth factor (pDGF). Other chemotactic factors include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor (alpha), fibroblast growth factor, platelet-derived endothelial cell growth factor, estradiols, insulin-like growth factor, nerve growth factor, bone growth/cartilage-inducing factor (alpha and beta), and matrix metallo proteinase inhibitors. Other cell response modifiers are the interleukins, interleukin inhibitors or interleukin receptors, including interleukin 1 through interleukin 10; interferons, including alpha, beta and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, DNA that encodes for the production of any of these proteins, antisense molecules, androgenic receptor blockers and statin agents.

Examples of bioactive agents include sirolimus, including analogues and derivatives thereof (including rapamycin, ABT-578, everolimus). Sirolimus has been described as a macrocyclic lactone or triene macrolide antibiotic and is produced by Streptomyces hygroscopicus, having a molecular formula of C₅₁H₇₉O₁₃ and a molecular weight of 914.2. Sirolimus has been shown to have antifungal, antitumor and immunosuppressive properties. Another suitable bioactive agent includes paclitaxel (Taxol) which is a lipophilic (i.e., hydrophobic) natural product obtained via a semi-synthetic process from Taxus baccata and having antitumor activity.

Other suitable bioactive agents include, but are not limited to, the following compounds, including analogues and derivatives thereof: dexamethasone, betamethasone, retinoic acid, vinblastine, vincristine, vinorelbine, etoposide, teniposide, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, melphalan, chlorambucil, ethylenimines and methylmelamines, alkyl sulfonates-busulfan, nitrosoureas, carmustine (BCNU) and analogs, streptozocin, trazenes-dacarbazinine, methotrexate, fluorouracil, floxuridine, cytarabine, mercaptopurine, thioguanine, pentostatin, 2-chlorodeoxyadenosine, cisplatin, carboplatin, procarbazine, hydroxyurea, mitotane, aminoglutethimide, estrogen, heparin, synthetic heparin salts, tissue plasminogen activator, streptokinase, urokinase, dipyridamole, ticlopidine, clopidogrel, abciximab, breveldin, cortisol, cortisone, fludrocortisone, prednisone, prednisolone, 6U-methylprednisolone, triamcinolone, triamcinolone acetonide, acetaminophen, etodalac, tolmetin, ketorolac, ibuprofen and derivatives, mefenamic acid, meclofenamic acid, piroxicam, tenoxicam, phenylbutazone, oxyphenthatrazone, nabumetone, auranofin, aurothioglucose, gold sodium thiomalate, tacrolimus (FK-506), azathioprine, mycophenolate mofetil, vascular endothelial growth factor (VEGF), angiotensin receptor blocker, nitric oxide donors, anti-sense oligonucleotides and combinations thereof, cell cycle inhibitors, mTOR inhibitors, and growth factor signal transduction kinase inhibitors. Another suitable bioactive agent includes morpholino phosphorodiamidate oligmer.

A comprehensive listing of bioactive agents can be found in The Merck Index. Thirteenth Edition, Merck & Co. (2001), the entire content of which is incorporated by reference herein. Bioactive agents are commercially available from Sigma Aldrich (e.g., vincristine sulfate). Additives such as inorganic salts, BSA (bovine serum albumin), and inert organic compounds can be used to alter the profile of bioactive agent release, as known to those skilled in the art.

In some embodiments, more than one active agent can be used. Specifically, co-agents or co-drugs can be used. A co-agent or co-drug can act differently than the first agent or drug. The co-agent or co-drug can have an elution profile that is different than the first agent or drug.

The phrase “therapeutically effective amount” is an art-recognized term. In some aspects, the term refers to an amount of the bioactive agent that, when incorporated into a medium of the invention, produces some desired effect at a reasonable benefit/risk ratio applicable to any medical treatment. The therapeutically effective amount can vary depending upon such factors as the condition being treated, the particular bioactive agent(s) being administered, the size of the patient, the severity of the condition, and the like. One of ordinary skill in the art can empirically determine the effective amount of a particular bioactive agent without necessitating undue experimentation.

The drug delivery media provide means to deliver bioactive agents from a variety of biomaterial surfaces. Biomaterials include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls, such as those polymerized from ethylene, propylene, styrene, vinyl chloride, vinyl acetate, vinyl pyrrolidone, and vinylidene difluoride. Examples of condensation polymers include, but are not limited to, nylons such as polycaprolactam, poly(lauryl lactam), poly(hexamethylene adipamide), and poly(hexamethylene dodecanediamide), and also polyurethanes, polycarbonates, polyamides, polysulfones, poly(ethylene terephthalate), poly(lactic acid), poly(glycolic acid), poly(lactic acid-co-glycolic acid), polydimethylsiloxanes, polyetheretherketone, poly(butylene terephthalate), poly(butylene terephthalate-co-polyethylene glycol terephthalate), esters with phosphorus containing linkages, non-peptide polyamino acid polymers, polyiminocarbonates, amino acid-derived polycarbonates and polyarylates, and copolymers of polyethylene oxides with amino acids or peptide sequences.

Certain natural materials are also suitable biomaterials, including human tissue such as bone, cartilage, skin and teeth; and other organic materials such as wood, cellulose, compressed carbon, and rubber. Other suitable biomaterials include metals and ceramics. The metals include, but are not limited to, titanium, stainless steel, and cobalt chromium. A second class of metals includes the noble metals such as gold, silver, copper, and platinum. Alloys of metals may be suitable for biomaterials as well, such as nitinol (e.g. MP35). The ceramics include, but are not limited to, silicon nitride, silicon carbide, zirconia, and alumina, as well as glass, silica, and sapphire. Yet other suitable biomaterials include combinations of ceramics and metals, as well as biomaterials that are fibrous or porous in nature.

The coatings of the invention are applied to a surface in a manner sufficient to provide a suitably durable and adherent coating on the surface. Typically, coatings are provided in a manner such that they are not chemically bound to the surface. Rather, the coatings can be envisioned as encapsulating the device surface. Given the nature of the association between the coating and the surface, it will be readily apparent that the coatings can be applied to virtually any surface material to provide a suitably durable and adherent coating. Moreover, in some embodiments, the surface can be suitably pretreated to enhance the association between the coating and the device surface.

In some embodiments, the polyelectrolyte coating is spray coated onto a surface of an implantable device as described herein. In other embodiments the coating is applied by immersing the surface into solutions of the polyanion and/or polycation.

The polyelectrolyte can be applied to any desired portion of a device surface. For example, in some embodiments, the entire surface of device is coated. In other embodiments, only a portion of the surface is coated.

As discussed above, the polyelectrolyte coatings of the present invention form either a hydrogel or blend depending on the polyanion arid polycation selected. In either case, certain embodiments provide for a spray coating technique that permits formation off multilayers, blends, or hydrogel coatings on the surface of a device.

Embodiments of the present invention can be used to apply coatings comprised of multiple polyelectrolyte components. Specifically embodiments of the present invention can be used to form coatings by separately delivering a first component and a second component to the surface of a medical device in a manner that limits or controls mixing of the components prior to application.

The term “coating solution”, as used herein, shall refer to a solution that is later atomized and sprayed to form a coating, or a part of a coating, and includes one or more polymers, one or more active agents, or both one or more polymers and one or more active agents. Coating solutions can also include other components such as solvents, stabilizers, salts, and the like.

The term “polymer solution”, as used herein shall refer to a coating solution that includes one or more polymers but not active agents. The term “active agent solution”, as used herein, shall refer to a coating solution that includes one or more active agents but not polymers. Both polymer solutions and active agent solutions can include other components such as solvents stabilizers, salts, and the like.

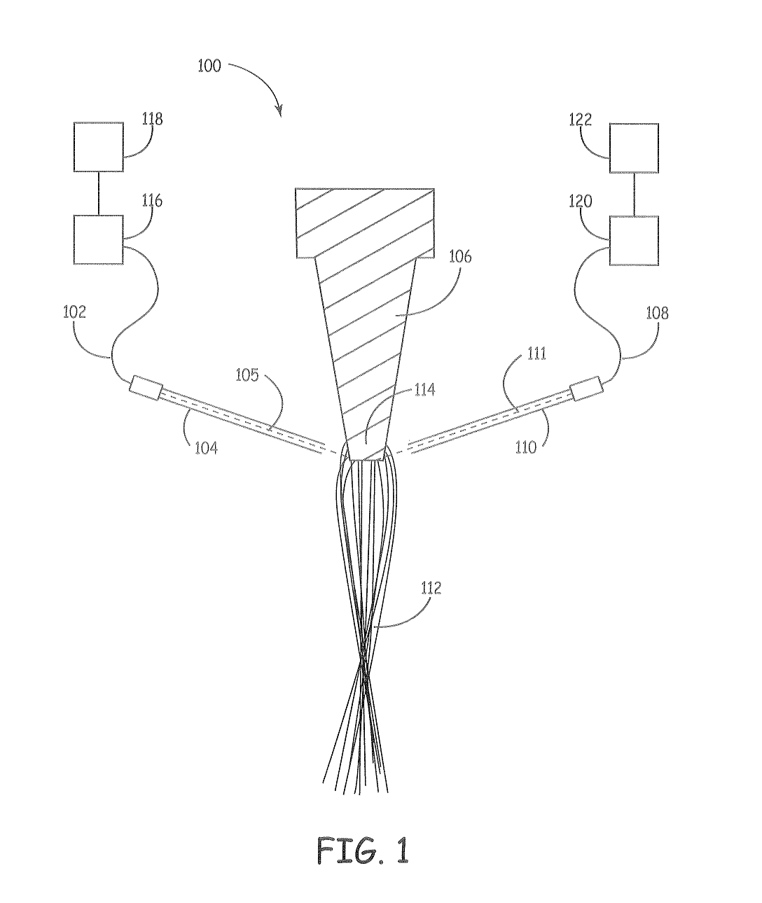

Some embodiments of the invention will now be described with reference to the figures. FIG. 1 shows a schematic side view of a coating apparatus 100 in accordance with an embodiment of the invention. A first solution supply line 102 connects to a first solution delivery conduit 104 that applies a first coating solution 105 onto the exterior surface of a nozzle 106. As an example, the first solution delivery conduit 104 may be made from hypodermic needle tube stock. The nozzle 106 has an atomization surface 114. The nozzle 106 can be an ultrasonic-atomization type spray nozzle (or ultrasonic nozzle).

Ultrasonic nozzles transmit vibrational energy to a liquid in an amount sufficient to atomize the liquid and form a spray of droplets. Ultrasonic nozzles are available commercially, such as from Sono-Tek, Milton, N.Y. Different types and sizes of ultrasonic nozzles may be used depending on the specific coating solutions used and the desired attributes of the spray stream generated. Ultrasonic nozzles may be designed to operate at specific frequencies. In an embodiment a 60 KHz ultrasonic nozzle can be used. The desired power level for operating the ultrasonic nozzle may depend on various factors including the size and design of the nozzle, the viscosity of the solution being used, the volatility of components in the solution being used, etc. In some embodiments the ultrasonic nozzle is operated at a power range of about 0.3 watts to about 3.0 watts. In an embodiment, the ultrasonic nozzle is operated at a power range of about 0.5 watts to about 1.5 watts. Exemplary ultrasonic nozzles are described in U.S. Pat. No. 4,978,067, the content of which is herein incorporated by reference.

The first solution supply line 102 is connected to a first pump 116 and a first solution supply reservoir 118. The first pump 116 can be set to deliver the first coating solution 105 at any desired rate. By way of example, the first pump 116 can be set to deliver the first coating solution 105 at a rate of from about 0.001 ml/minute to about 20 ml/minute. In an embodiment, the first pump 116 delivers the first coating solution 105 at a rate of about 0.01 ml/minute to about 1.0 ml/minute. The rate at which the first pump 116 delivers the first coating solution 105 can be varied during the coating process. The first pump 116 can be controlled by a controller unit (not shown). The first coating solution 105 is converted into a spray stream 112 by the nozzle 106. In an embodiment, the first coating solution 105 is atomized by the nozzle 106.

A second solution supply line 108 connects to a second solution delivery conduit 110 which applies the second coating solution 111 onto the exterior surface of nozzle 106. As an example, the second solution delivery conduit 110 may be made from hypodermic needle tube stock. The second solution supply line 108 is connected to a second pump 120 and a second solution supply reservoir 122. The second pump 120 can be set to deliver the second coating solution 111 at any desired rate. By way of example, the second pump 120 can be set to deliver the second coating solution 111 at a rate of from about 0.001 ml/minute to about 20 ml/minute. In an embodiment, the second pump 120 delivers the second coating solution 111 at a rate of about 0.01 ml/minute to about 1.0 ml/minute. The rate at which the second pump 120 delivers the second coating solution 111 can be varied during the coating process. The second pump 120 can be controlled by a controller unit (not shown). The second coating solution 111 is converted into a spray stream 112 by the nozzle 106. In an embodiment, the second coating solution 111 is atomized by the nozzle 106.

The pumping rate of the first pump 116 and the pumping rate of the second pump 120 can be the same or different. As an example, the pumping rates of the pumps can be manipulated so that more of one coating solution (105 or 111) is applied than the other. The pumping rate of the first pump 116 and the pumping race of the second pump 120 may be constant or variable over time.

The first coating solution 105 and the second coating solution 111 may be applied to the nozzle 106 either simultaneously or sequentially. In an embodiment, first coating solution 105 and second coating solution 111 are applied to the nozzle 106 simultaneously. In some embodiments, the first coating solution 105 and the second coating solution 111 do not contact each other until after they are applied to the surface of the nozzle 106.

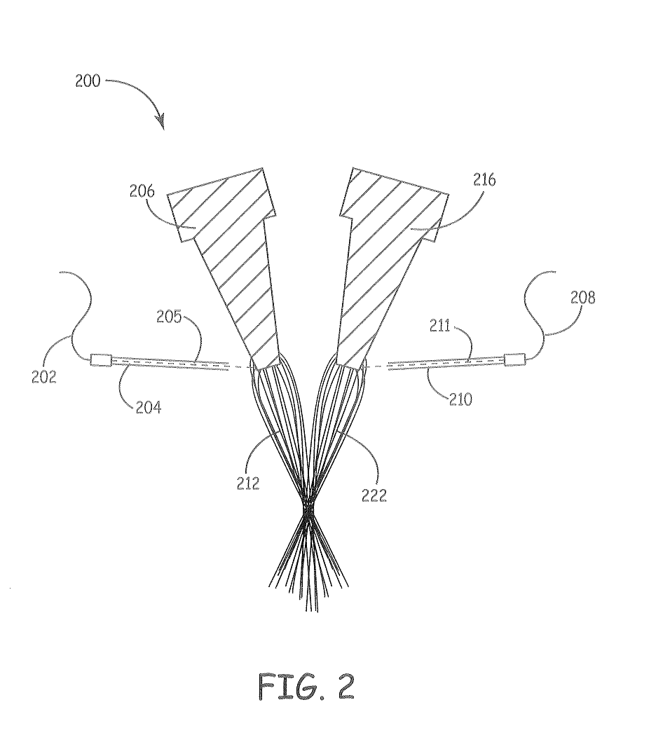

FIG. 2 describes another embodiment of the apparatus. In this embodiment, there is a first nozzle 206 and a second nozzle 216. A first solution supply line 202 connects to a first solution delivery conduit 204 that applies the first coating solution 205 onto the first nozzle 206. The first solution supply line 202 is connected to a pump (not shown) and a first solution supply reservoir (not shown). The pump can be set to deliver the first coating solution 205 at any desired rate. The first coating solution 205 is converted into a spray stream 212 by the nozzle 206.

A second solution supply line 208 connects to a second solution delivery conduit 210 that applies the second coating solution 211 onto the second nozzle 216. The second solution supply line 208 is connected to a pump (not shown) and a second solution supply reservoir (not shown). The pump can be set to deliver the second solution at any desired rate. The second coating solution 211 is converted into a spray stream 222 by the second nozzle 216. In this embodiment, the first coating solution 205 and the second coating solution 211 do not contact each other until their respective spray streams 212 and 222 meet.

In certain embodiments, the apparatuses described in FIGS. 1 and 2 are used to produce polyelectrolyte hydrogel coatings. In other embodiments, polyelectrolyte blend coatings are produced. In yet other embodiments, polyelectrolyte multilayers are produced.

As is apparent, any coating can be applied using either of the embodiments shown in FIGS. 1 and 2. For example, a polyelectrolyte coating can be applied with the embodiment in FIG. 1. In these embodiments, the polyanion and polycation are sprayed from the same nozzle. Alternately a polyelectrolyte coating can be applied with the embodiment depicted in FIG. 2. In these embodiments, the polyanion and polycation are sprayed from the separate nozzles. In both cases, however, the polyanion and polycation are kept separate from each other until the moment they are sprayed on a surface.

In embodiments utilizing the apparatuses of FIGS. 1 and 2, the first coating solution 105, 205 comprises the polyanion and the second coating solution 111, 211 comprises the polycation. As will be apparent, the first coating solution can comprise the polycation and the second coating solution can comprise the polyanion.

In some embodiments, the first coating solution 105, 205 additionally comprises the bioactive agent while in other embodiments the second coating solution 111, 211 additionally comprises the bioactive agent, in other embodiments, both the first 105, 205 and second 111, 211 coating solutions additionally comprise the bioactive agent. In yet other embodiments, neither of the coating solutions 105, 205 or 111, 211 comprise a bioactive agent.

The type of coating produced by either of the above apparatuses is dependent on the polyanion and or polycation selected and the method by which they are applied. For example, polyelectrolyte hydrogel coatings can be obtained by providing polyanion and polycation materials that interact to form a gel (as previously discussed) as the first 105, 205 and second 111, 211 coating solutions. In some embodiments, the hydrogel is formed by simultaneously spraying the polyanion and polycation oil the surface. In other embodiments, the polyanion and polycation are spayed sequentially so that the layers interact on the surface of the device to form the hydrogel.

In other embodiments, polyelectrolyte blend coatings are produced. As with hydrogels, blended coatings can be applied simultaneously or sequentially. It will be appreciated that in embodiments where blends or hydrogels are produced, no surface pretreatment to produce a charged surface is required since the adherence of the coating to the surface is not dependent on ionic interactions between the coating and the surface.

In other embodiments, polyelectrolyte multilayer coatings are produced. In these embodiments the first 105, 205 and second 111, 211 coating solutions are applied sequentially and each comprise either a polyanion or polycation. In these embodiments, a first layer is applied and dried. Thereafter, a second layer of oppositely polyelectrolyte is applied and allowed to dry. The application and drying steps are repeated until the desired number of layers is obtained.

As is known to those skilled in the art, application of PEM coatings requires pretreatment of the surface to which the coating is applied. That is, the surface must be treated so that it becomes charged. The first layer of the PEM then adheres to the surface by means of ionic interactions between the charge on the surface and the charge on the polyelectrolyte. However, in embodiments encompassed by this disclosure, PEM coatings can be applied without the need for surface pretreatment, for example, in embodiments where photogroups are included. Such use of photogroups will now be described with more detail.

In embodiments where photo-polyelectrolytes are selected, the presence of photogroups may be used to attach the polyelectrolyte coatings to a surface. The first photo-polyelectrolyte is attached to the surface of a medical device via the photoreactive groups by methods known to those skilled in the art. The coating is then made by simply contacting the device surface with the photopolymer coupled to a polyelectrolyte of the opposite charge. For example, in one embodiment a photo-polycation is selected. The photo-polycation is contacted with a surface and irradiated thereby coupling the polycation to the surface. The polyelectrolyte coating is formed by contacting the surface (with the coupled photo-polycation) with a polyanion. Electrostatic interactions between oppositely charged polyelectrolytes create an insoluble blend, hydrogel, or PEM upon the surface.

In any of the embodiments the bioactive agent can be included with the polyanion, the polycation, or alternatively, both the polyanion and polycation. Some bioactive agents carry a net charge or are associated with a charged molecule. Even non-charged bioactive agents can be modified so that they are charged. For example, a neutral bioactive agent can be non-covalently coupled to a charged species. Thus, in some embodiments, the bioactive agent carries a net charge, either directly are through association with other molecules or species. In these embodiments, the bioactive agent can be provided with the polyelectrolyte of like charge. For example, a positively charged bioactive agent can be provided with the polycation. Alternately, a negatively charged bioactive agent can be provided with the polyanion.

As will be appreciated, in embodiments where multiple layers of the coating are produced, bioactive agent can be provided in all layers or alternately in only a selected number of layers. For example in PEM coating embodiments, the bioactive agent can be provided in one, more than one, or all of the polyanion or polycation layers. Alternately, bioactive agent can be provided in one, more than one, or all of both polyanion and polycation layers. In yet other alternatives, bioactive agent is not provided in any of the layers, but rather is provided as intermediate layer(s) sandwiched between the layers.

Alternately, the bioactive agent can be provided in an additional layer that is provided under the polyelectrolyte coating. Alternately, the bioactive agent can be provided in a top coat, which is applied over the polyelectrolyte coating. The topcoats can be comprised of polyelectrolyte materials or alternately of non-polyelectrolyte materials.

In other embodiments, the bioactive agent is incorporated after the polyelectrolyte bioactive agent delivery medium is produced. Incorporation can be achieved by, for example, simple diffusion. In other embodiments, the bioactive agent can be incorporated electrophoretically.

In some embodiments, the surface of some biomaterials can be pretreated (e.g., with a silane and/or Parylene™ coating composition in one or more layers) in order to alter the surface properties of the biomaterial. For example, in various embodiments of the present invention a layer of silane may be applied to the surface of the biomaterial followed by a layer of Parylene™. Parylene™ C is the polymeric form of the low-molecular-weight dimer of para-chloro-xylylene. Silane and/or Parylene™ C (a material supplied by Specialty Coating Systems (Indianapolis)) can be deposited as a continuous coating on a variety of medical device parts to provide an evenly distributed, transparent layer.

Also, as previously described above, the surface to which the medium is applied can itself be pretreated in other manners sufficient to improve attachment of the composition to the underlying (e.g., metallic) surface. Additional examples of such pretreatments include photografted polymers, epoxy primers, polycarboxylate resins, and physical roughening of the surface. It is further noted that the pretreatment compositions and/or techniques may be used in combination with each other or may be applied in separate layers to form a pretreatment coating on the surface of the medical device.

In some embodiments, the surfaces can be pretreated to provide a tie-layer. Tie-layers have been discussed in detail in U.S. Pat. Nos. 6,254,634 and 6,706,408, the contents of which are hereby incorporated by reference.

The bioactive agent delivery medium of the present invention can be used in combination with a variety of devices, including those used on a temporary, transient, or permanent basis upon and/or within the body.

Coatings of this invention can be used to coat the surface of a variety of implantable devices, for example: drug-delivering vascular stents (e.g., self-expanding stents typically made from nitinol, balloon-expanded stents typically prepared from stainless steel); other vascular devices (e.g., grafts, catheters, valves, artificial hearts, heart assist devices); implantable defibrillators; blood oxygenator devices (e.g., tubing, membranes); surgical devices (e.g., sutures, staples, anastomosis devices, vertebral disks, bone pins, suture anchors, hemostatic barriers, clamps, screws, plates, clips, vascular implants, tissue adhesives and sealants, tissue scaffolds); membranes; cell culture devices; chromatographic support materials; biosensors; shunts for hydrocephalus; wound management devices; endoscopic devices; infection control devices; orthopedic devices (e.g., for joint implants, fracture repairs); dental devices (e.g., dental implants, fracture repair devices), urological devices (e.g., penile, sphincter, urethral, bladder and renal devices, and catheters); colostomy bag attachment devices; ophthalmic devices (e.g. ocular coils); glaucoma drain shunts; synthetic prostheses (e.g., breast); intraocular lenses; respiratory, peripheral cardiovascular, spinal, neurological, dental, ear/nose/throat (e.g., ear drainage tubes); renal devices; and dialysis (e.g., tubing, membranes, grafts).

It is important to note that the local delivery of combinations of bioactive agents may be utilized to treat a wide variety of conditions utilizing any number of medical devices, or to enhance the function and/or life of the device. Essentially, any type of medical device may be coated in some fashion with one or more bioactive agents that enhances treatment over use of the individual use of the device or bioactive agent.

The coating compositions of the present invention can be applied to the device in any suitable fashion (e.g. the coating composition can be applied directly to the surface of the medical device or alternatively to the surface of a surface-modified medical device, by dipping, spraying, ultrasonic deposition, or using any other conventional technique). The suitability of the coating composition for use on a particular material, and in turn, the suitability of the coated composition can be evaluated by those skilled in the art, given the present description.

In one such embodiment, for instance, the coating comprises at least two non-identical layers. For instance, a base layer may be applied having bioactive agent(s) alone, or together with or without one or more of the polymer components, after which one or more topcoat layers are coated, each with either first and/or second polymers as described herein, and with or without bioactive agent. These different layers, in turn, can cooperate in the resultant composite coating to provide an overall release profile having certain desired characteristics. In various embodiments, the composition is coated onto the device surface in one or more applications of a single composition that includes first and second polymers, together with bioactive agent. While in other embodiments, the composition is coated in one or more applications as more than one composition that includes individual polymers. However, as previously suggested a pretreatment layer or layers may be first applied to the surface of the device, wherein subsequent coating with the composition may be performed onto the pretreatment layer(s). The method of applying the coating composition to the device is typically governed by the geometry of the device and other process considerations. The coating is subsequently cured by evaporation of the solvent. The curing process can be performed at room or elevated temperature, and optionally with the assistance of vacuum and/or controlled humidity.

It is also noted that one or more additional layers may be applied to the coating layer(s) that include bioactive agent. Such layer(s) or topcoats can be utilized to provide a number of benefits, such as biocompatibility enhancement, delamination protection, durability enhancement, bioactive agent release control. In one embodiment the topcoat may include one or more of the polyanion, polycation, and/or additional polymers described herein with or without the inclusion of a bioactive agent, as appropriate to the application. In some embodiments the topcoat includes a second polymer that is a poly(alkyl(meth)acrylate). An example of a poly(alkyl(meth)acrylate) includes poly(n-butyl methacrylate). In another embodiment, the polyanion or polycation polymers could further include functional groups (e.g. hydroxy, thiol, methylol, amino, and amine-reactive functional groups such as isocyanates, thioisocyanates, carboxylic acids, acyl halides, epoxides, aldehydes, alkyl halides, and sulfonate esters such as mesylate, tosylate, and tresylate) that could be utilized to bind the topcoat to the adjacent coating composition. In another embodiment of the present invention one or more of the pretreatment materials (e.g. Parylene™) may be applied as a topcoat. Additionally, biocompatible topcoats (e.g., but not limited to, heparin, collagen, extracellular matrices cell receptors) may be applied to the coating composition of the present invention. Such biocompatible topcoats may be adjoined to the coating composition of the present invention by utilizing photochemical or thermochemical techniques known in the art. Additionally, release layers may be applied to the coating composition of the present invention as a friction barrier layer or a layer to protect against delamination. Examples of biocompatible topcoats that may be used include those disclosed in U.S. Pub. Nos. US 2003-0232087 and US 2006-0147491, the contents of which are incorporated by reference.

In use, the hydrogel media are either coated on device surfaces, used to fill hollow interior devices or directly implanted. The blend and PEM media are typically used as coatings on medical device surfaces. In any case, the media are provided with a therapeutically effective amount of bioactive agent and placed with a patient or subject at a desired implantation site. At the implant site, the bioactive agent is delivered via either simple diffusion of the agent out of the medium or is released as the medium breaks down as is the case when biodegradable materials are selected.

In preferred aspects, the active agent delivery media can provide controlled release of bioactive agent to thereby provide a therapeutically effective dose of the bioactive agent for a sufficient time to provide the intended benefits.

The invention may be better understood by reference to the following non-limiting examples. Table 2 is a list of abbreviations of terms used in Table 1 and in the examples.

TABLE 2
List of Abbreviations

| | |
|---|---|
| PA | Polyacrylamide |
| AMPS | 2-Acrylamido-2-methyl-1-propanesulfonic acid |
| APMA | N-(3-aminopropyl) methacryl amide |
| PEG | Polethylene glycol |
| MA | Methacrylic acid |
| AEM | Aminoethylmethacrylate |
| PVP | Polyvinylpyrrolidone |
| EAC | Epsilon aminocaproic acid |
| MAPTAC | Methacrylamidopropyl triethylammonium chloride |
| DMA | Dimethylacrylamide |
| HPMA | Hydroxypropoylmethacrylamide |
| MAA | Methacrylic acid |
| DiBBE | Dibenzoylbenzyl ether |
| DHBA | Dihydroxybenzoic acid |
| NOS | N-oxysuccinimide |
| mL | Milliliter |
| mg | Milligram |
| nm | Nanometer |
| ug | Microgram |
| μm | Micrometer |
| PSS | Polystyrene sulfonate |
| PAH | Poly(allyl amine hydrochloride) |
| BSA | bovine serum albumin |
| LHRH | Lutein hormone release hormone |
| pDNA | Plasmid DNA |
| PEI | Polyethyleneimine |
| DNA | Deoxyribonucleic acid |

### EXAMPLE 1

### Preparation of PSS/PAH Blend Coatings

Separate solutions of PAH and PSS were prepared in water to a final concentration of 30 mg/mL. The solutions were passed through 0.45 μm filters. The solutions were coated on stainless steel coronary stents with an ultrasonic spraycoating system. The system was configured with two independent solutions flowing to the sprayhead. This, in combination with independent syringe pumps used to feed the sprayhead, permitted spraying each solution alone or simultaneously. The PSS and PAH solutions were loaded into separate syringes in the spray system.

Two spraying methods, dual spray and the alternate spray method were utilized. In the dual spray method, PSS and PAH solutions were simultaneously delivered to the nozzle and thus to the surface of the substrate as well. Without intending to be bound by theory, it is theorized that in the dual spray method, some mixing of PSS and PAH occurred on the nozzle with further mixing occurring on the surface of the substrate. In the alternate spray method, alternate layers of PSS and PAH were applied with a single nozzle. Three layers of each PSS and PAH were applied. Without intending to be bound by theory, it is theorized that mixing of PSS and PAH occurred on the surface of the substrate in the alternate spray method.

The stents were dried under a flow of nitrogen for 16 h and the coating accessed by microscopy and weighed. Tenacity of the coatings was evaluated by submerging the coated stents into an aqueous environment comprising PBS for 12 days, drying reweighing, and calculating the percent mass loss.

Tenacity studies indicate a robust coating with an approximate mass loss of 20%. These results suggest that the polymers blended during the coating process to produce insoluble polyanion-polycation complex.

### EXAMPLE 2

### PSS/PAH Blend Coatings Containing a Small Molecule Hydrophilic Drug Mimic

Coated stents were prepared according to Example 1 except the PSS solution was prepared at a concentration of 10 mg/mL and calcein was added at a concentration of 20 mg/mL. The flow rate of the two solutions was adjusted such that the final coating contained 33 wt % calcein and 67 wt % polymer.

### EXAMPLE 3

### Elution of Calcein from PSS/PAH Polyelectolyte Blend Coatings

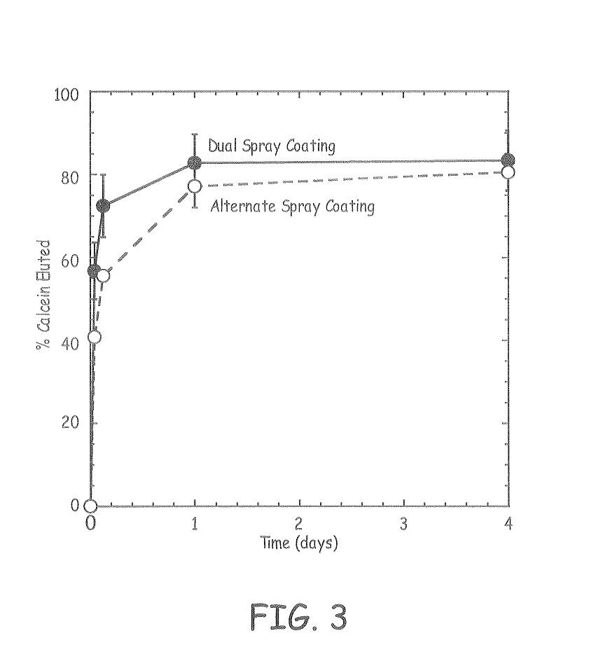

Stents coated with PSS/PAH containing calcein were prepared according to Example 2. The elution of calcein from the coated stents was accessed by placing the stents in phosphate-buffered saline, pH 7.4 at 37° C. Presence of calcein in the saline was monitored by detection of fluorescence at ex. 494 nm, em. 517 nm. FIG. 3 depicts the elution profile of calcein from PSS/PAH coatings produced by the dual and alternate spray methods.

### EXAMPLE 4

### Elution of BSA and LHRH from PSS/PAH Polyelectrolyte Blend Coatings

Solutions of PAH and PSS were made in water to a final concentration of 20 mg/mL. LHRA or BSA was added to the PSS and PAH solutions to a final concentration of 10 mg/mL. Stents were coated with PSS/PAH containing either BSA or LHRH according to Example 1.

The elution of BSA or LHRH from the coated stents was assessed by placing the stents in phosphate-buffered saline, pH 7.4 at 37° C. for one hour and measuring the amount of either BSA or LHRH in the saline using the BCA protein concentration kit available from Sigma-Aldrich.

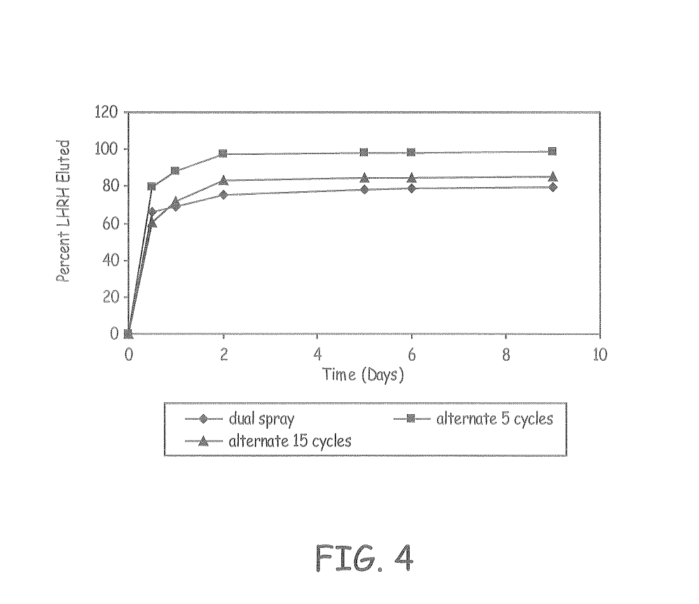

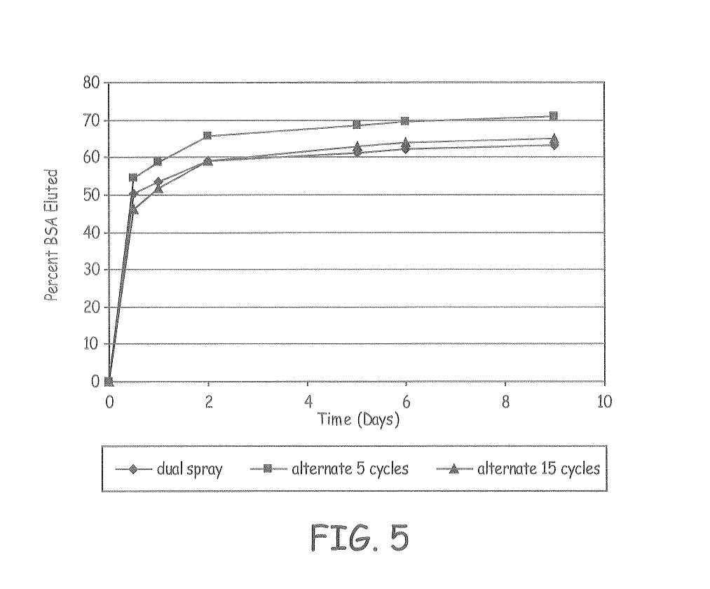

The elution profiles of LHRH and BSA from PSS/PAH coatings are depicted in FIGS. 4 and 5, respectively.

### EXAMPLE 5

### Evaluation of Blend Coatings Formed from Natural Polyelectrolytes

Six separate solutions containing one each of Gelatin A, Gelatin B, polylysine, DNA, chitosan, and heparin are prepared in water by dissolving the polymer to a final concentration of 20 mg/mL. Fluorescamine labeled gentamycin, BSA, or LHRH are dissolved in the solutions to a final concentration of 10 mg/mL. The solutions are passed through 0.45 μm filters and spray coated onto stainless steel coronary stents according to the method set out in Example 1.

The durability of the coatings is evaluated according to procedures set out in Example 3. Elution of gentamycin, BSA, or LHRH is determined according to the procedures set out in Example 4. Elution of gentamycin is evaluated according to the fluorescence procedures set out in Example 1.

### EXAMPLE 6

### Evaluation of Coatings for Use as DNA Delivery Vehicles

Herring DNA is dissolved in low ionic strength PBS (5 mM, 0% NaCl) and fluorescently labeled pDNA added to a final concentration of 20 mg/mL herring DNA, 10 mg/mL pDNA. Solutions of 20 mg/mL PEI (linear or branched) are prepared in water. The two solutions are spray coated onto the stents according to the procedures set out in Example 1. To form pDNA/PEI polyplexes, fluorescently labeled pDNA is incubated with PEI in deionized water. The polyplexes are added to a 20 mg/mL water solution of PEI or PAH. The polycation/polyplex solution is cosprayed with a 20 mg/mL herring DNA solution onto stents according to the spraying methods of Example 1.

The durability of the coating is tested according to procedures set out in Example 4. To evaluate the controlled release properties of the stents, the stents are soaked in buffer for a variety of time periods and the elutant evaluated for presence of pDNA by detection of fluorescence.

The integrity of the eluted pDNA can be evaluated by loading eluted pDNA samples onto agarose gels and electrophoretically separating the pDNA product. To determine the efficiency of cell transfection of the eluted pDNA, the pDNA eluted from the stent is incubated with immortal cell lines and the amount of pDNA taken up by the cells determined.

### EXAMPLE 7

### Preparation of a Polycationic Maltodextrin

Polycationic maltodextrin is prepared by dissolving 5.0 g maltodextrin (DE 4-7, 30.5 mmeq of hydroxyl groups), 4.7 g betaine hydrochloride (30.6 mmol), 0.5 g DMAP (4-dimethylaminopyridine, 4.1 mmol), and 10.0 gNHS (N-hydroxysuccinimide, 8.7 mmol) in 20 mL DMSO. To the solution, 7.6 g of DIC (diisopropylcarbodiimide) is added and the reaction stirred overnight. The reaction is added to 1.0 L of water. The water solution is concentrated, difiltered, and lyophilized to the give the product.

### EXAMPLE 8

### Preparation of Polyanionic Maltodextrin

Polyanionic maltodextrin is prepared by dissolving 5.0 g of maltodextrin (DE-47, 30.5 mmeq of hydroxyl groups and 0.5 g of DMAP (4-dimethylaminopyridine, 4.1 mmol) in 15 mL DMSO. A second solution is made by dissolving 6.2 g of sodium solfosuccinic anhydride (30.5 mmol) in 10 mL of DMSO. The solutions are mixed and stirred overnight. The reaction is added to 1.0 L of water. The water solution is concentrated, difiltered, and lypholized to give the product.

### EXAMPLE 9

### Preparation of a Maltodextrin Hydrogel

Water solutions of polycationic and polyanionic maltodextrin are prepared according to Examples 7 and 8. A polyelectrolyte hydrogel is formed by mixing the water solutions of polycationic and polyanionic maltodextrin together.

### EXAMPLE 10

### Preparation of a PEI/Alginic Acid Hydrogel

A 10% solution of PEI was prepared in water. A solution of alginic acid was prepared in water to a final concentration of 400 mg/mL. The PEI and alginic acid solutions were mixed together and 40 mg of rabbit IgG was added. Gels were allowed to set up overnight at 28° C.

The PEI/alginic acid formed a clear gel that became cloudy upon addition of rabbit IgG, evidencing the distribution of the IgG throughout the gel matrix. Addition of protein appeared to accelerate the gel set up process. By visual inspection, PEI and alginic acid appeared to form firm and durable hydrogels.

While specific embodiments of the present invention have been described, it should be understood that various changes, adaptations, and modifications can be made without departing from the spirit of the invention and the scope of the appended claims.

We claim:

1. A composition for coating at least a portion of a surface of a medical device, the coating composition comprising at least one bioactive agent, a first polymer component comprising a polyanionic polymer and a second polymer component comprising a polycationic polymer, wherein the first and second polymer components are selected so as to form a coating selected from the group consisting of blended coatings and hydrogel coatings.

2. The coating composition of claim 1, wherein the first and second polymer components are synthetic polymers.

3. The coating composition of claim 1, wherein the first and second polymer components are degradable polymers.

4. The coating composition of claim 1, wherein the first polymer component comprises poly (styrene sulfonate) and the second polymer component comprises poly(allyl amine hydrochloride).

5. The coating composition system of claim 1, wherein the first, second, or both polymer components further comprise at least one photoreactive group.

6. The coating composition system of claim 1, wherein the bioactive agent is provided with the polyanionic polymer component, the polycationic polymer component, or both.

7. The coating composition system of claim 1, wherein the bioactive agent is selected from the group consisting of hydrophilic drugs, hydrophobic drugs, peptides, proteins, or nucleic acids.

8. The coating composition of claim 1, wherein the relative ratios of the first and second polymer components are selected so to achieve a desired pH of a microenvironment of a coating formed by the coating composition.

9. The coating composition of claim 1, wherein the first polymer component comprises poly(ethyleneimine) and the second polymer component comprises alginate.

10. An implantable medical device comprising; a surface coated with a polyelectrolyte coating and a bioactive agent, wherein the polyelectrolyte coating comprises a first polyanionic polymer component and a second polycationic polymer component, wherein the first and second polymer components intermingle and do not form a multilayer structure.

11. The implantable medical device of claim 10, wherein the first and second polymer components are selected so as to form a hydrogel.

12. The implantable medical device of claim 10, wherein the first polyanionic polymer component comprises alginate, the second polycationic polymer comprises poly(ethyleneimine).

13. The implantable medical device of claim 10, wherein the first polymer component, the second polymer component, or both are derivatized with at least one photoreactive group.

14. The implantable medical device of claim 13, wherein the photogroups are activated so that covalent bonds are formed between photoreactive groups on the first and second polymer components, the surface of the medical device, or both.

15. The implantable medical device of claim 11, wherein the relative ratio of polyanionic polymer to polycationic polymer are adjusted so as to achieve a desired pH range in a microenvironment of the hydrogel coating.

16. The implantable medical device of claim 10, wherein the first and second polymer components are selected so as to form a blended coating.

17. The implantable medical device of claim 16, wherein the first polymer component, the second polymer component, or both further comprise at least one photoreactive group.

18. The implantable medical device of claim 17, wherein the photogroups are activated so that covalent bonds are formed between photoreactive groups on the first and second polymer components, the surface of the medical device, or both.

19. The implantable medical device of claim 16, wherein the relative ratio of polyanionic polymer to polycationic polymer are adjusted so as to achieve a desired pH range in a microenvironment of the blended coating.

20. The implantable medical device of claim 15, wherein the first polyanionic polymer component comprises poly(styrene sulfonate) and the second polycationic polymer component comprises poly (allyl amine hydrochloride).

21. A combination comprising an implantable medical device and a coating composition system for providing a polyelectrolytic coating on a surface of the medical device in a manner that permits the coated surface to release a bioactive agent over time when implanted in vivo, the composition system comprising at least one bioactive agent, a first polyanionic polymer, and a second polycationic polymer.

22. The combination of claim 21, wherein the first and second polymer components are selected so as to form a hydrogel.

23. The combination of claim 21, wherein the first polymer component comprises alginate and the second polymer component comprises poly(ethyleneimine).

24. The combination of claim 21, wherein the first polymer component, the second polymer component, or both further comprise at least one photoreactive group.

25. The combination of claim 21, wherein the first and second polymer components are selected so as to form a blend.

26. The combination of claim 24, wherein the first polymer component comprises poly(allyl amine hydrochloride) and the second polymer component comprises poly (styrene sulfonate).

27. The combination of claim 21, wherein the first polymer component, the second polymer component, or both further comprise at least one photoreactive group.

28. An implantable polyionic hydrogel composition comprising a polyanionic polymer component, a polycationic polymer component, and at least one bioactive agent.

29. The polyionic hydrogel composition of claim 28 wherein the hydrogel is provided as a three dimensional matrix filling in at least a portion of a hollow three dimensional space of an implantable medical device.

30. The polyionic hydrogel composition of claim 28, wherein the hydrogel is provided as an implantable three dimensional matrix.

31. The polyionic hydrogel of claim 30, wherein the implantable three dimensional matrix is formed in situ.

32. The polyionic hydrogel composition of claim 28, wherein the polyanionic component comprises alginate and the polycationic component comprises poly(ethyleneimine).

33. A method for applying a polyionic coating to a surface, the method comprising the steps of: providing a polyanionic coating solution in a first reservoir and a polycationic coating solution in a second reservoir, wherein the first reservoir feeds a first nozzle and the second reservoir feeds a second nozzle; and applying the first and second coating solutions to the surface via the first and second nozzles.

34. The method according to claim 33, wherein the first and second coating solutions are simultaneously fed to the first and second nozzles.

35. The method according to claim 33, wherein the first and second coating solutions are sequentially fed to the first and second nozzle so that only one nozzle is applying coating solution at given time.

36. The method according to claim 33 wherein the first, second, or both coating solutions further comprises a bioactive agent.

37. The method according to claim 33, wherein the bioactive agent carries a net charge and is provided with the coating solution comprising the same net charge.

38. A method for applying a polyionic coating to a surface, the method comprising the steps of: providing a polyanionic coating solution in a first reservoir and a polycationic coating solution in a second reservoir, wherein the first and second reservoir feeds a first nozzle; and applying the first and second coating solutions to the surface via the first nozzle.

39. The method according to claim 38, wherein the first and second coating solutions are simultaneously fed to the first nozzle.

39. The method according to claim 38, wherein the first, second, or both coating solutions further comprise a bioactive agent.

40. The method according to claim 39, wherein the bioactive agent carries a net charge and is provided with the coating solution comprising the same net charge.
